(19) United States
(12) Patent Application Publication
(10) Pub. No.: US 2003/0078451 A1
(43) Pub. Date: Apr. 24, 2003

(54) Catalytic solution system for manufacturing terephthalic acid and a method for same

(75) Inventor: David Yen, Tao-Yuan Hsien (TW)

Correspondence Address: TROXELL LAW OFFICE PLLC, SUITE 1404, 5205 LEESBURG PIKE, FALLS CHURCH, VA, 22041, US

(73) Assignee: Mechema Chemicals International Corp.

(21) Appl. No.: 10/188,816

(22) Filed: Jul. 5, 2002

Related U.S. Application Data
(63) Continuation-in-part of application No. 09/983,314, filed on Oct. 24, 2001, now abandoned.

(51) Int. Cl. C07C 51/265

(52) U.S. Cl. 562/416

(57) ABSTRACT

A catalytic solution system for manufacturing terephthalic acid is disclosed and its method. The system uses a Co—Mn—Br acetate solution (CMB) that is applied in an oxidation reaction, when the weight ratio of each component is (Co %×2.7)+(Mn %×2.9 %)>Br %. CoBr₂ and MnBr₂ are introduced as a source of Br for the CMB solution. Compensation for any shortage of Co and Mn, after such a supplement, can be made from cobalt acetate and manganese acetate without using highly corrosive HBr. The present invention provides a method of preparing a catalyst for manufacturing terephthalic, wherein, in the stirring action of para-xylene with a gas containing oxygen, the above catalytic solution system is used.

This application is a continuation-in-part application of U.S. application Ser. No. 09/983,314, which was filed on Oct. 24, 2001.

### BACKGROUND OF THE INVENTION

1. Field of the Invention

The present invention relates to a catalytic solution system for manufacturing terephthalic acid and a method for same. Specifically, the invention relates to an acetate solution of Co, Mn, and Br (identified as “CMB”) wherein Co, Mn, and Br are replenished according to a weight ratio of each component is (Co %×2.7)+(Mn %×2.9%) to Br %.

2. Description of the Background Art

Purified terephthalic acid (PTA) is widely used as the raw material for various industrial processes. For example, the raw material of polyester for producing clothes is polyethylene terephthalate (PET). In this industrial process, purified terephthalic acid (PTA) and ethylene glycol (EG) are used in esterification and polycondensation reactions to obtain PET.

Purified terephthalic acid (PTA) is made by the chemical reaction of para-xylene (PX) with O₂. In this reaction the atoms of the oxygen molecules are in the state of triplet spin. These atoms will not interact with the carbon atoms in the hydrocarbon molecules (CPX), which are in the state of single spin, in order to undergo chemical oxidation. Therefore, at least one transition metal (Co, Mn, Cr, Ni, Cu, etc.) must be introduced into the process to form complex salts. These complex salts enable the ions of the metal to adsorb oxygen molecules easily through the combining force of a coordination/complexing field.

The basic states of those atoms are changed from a triple spin or “+3” charge to a single spin or “+” charge in which catalyzation starts the oxidation reaction of para-xylene (PX). The reaction is a spontaneous oxidation. For example, only an adequate concentration of a free radical can initiate the chemical reaction and then continuously enlarge the amplitude of oxidation of the PX by its chain reaction. However, the transition metal, cobalt, which starts the reaction, is also oxidized into Co⁺³ and is subjected to attack by acetic acid and PX under the high oxidation temperatures. These compounds will further decompose to carbon dioxide, methyl acetate, benzene m-tricarbonic acid, benzene carbonic acid, phenols, and diphenyl, etc. This decomposition adds to the cost of the process and degrades the quality of the product of the PTA process. The ions of Co⁺³ required for initiating the reaction are not required in a high concentration. If the ions are overly accumulated, the degradation phenomena will be greatly amplified.

Improvement of the process is achieved by using a suitable coordination radical for complexing. Such a radical reduces the half-life of Co⁺³ in the acetic acid. The radical also performs as the initiator of the PX oxidation. Bromide is a most suitable compound for combining with the cobalt salt. The concentration of acetic acid is utilized to control the selectivity of the oxidation of PX. (Partenheimer et al., U.S. Pat. No. 5,081,290.) Typically, Co, Mn, Br, etc., are used as the catalytic ions for achieving the best reaction rate and the best selectivity for product quality in these processes.

Another concern of PTA process is the maintenance of corrosion metals in the lowest possible concentrations. An increase in concentration in the reaction medium of corrosion metals, which act as catalysts for Friedel-Crafts reactions, significantly contributes to the side reactions in the PTA process.

The PTA catalyst for oxidation reactions of the background was normally derived from solid cobalt compounds, solid manganese compounds, or liquid bromine compounds. PTA manufacturers then had to mix and dissolve these compounds in a preparation tank to a desired concentration.

Cobalt acetate crystals and manganese acetate crystals often form blocks because of dehydration. These compounds are extremely inconvenient to store. Also the poor solubility of these solids requires significant manpower and time to produce a specific catalyst concentration before it can be injected into the PTA process. A ready-made solution of cobalt acetate and manganese acetate has been gradually introduced to replace the solid compounds used by manufacturers of PTA. These solutions save both catalyst users' and suppliers' operating costs incurred from crystallizing, separating, drying, packing, and re-dissolving procedures required with the solid compounds. Therefore, in using a solution of cobalt manganese acetate (CMA), the manufacturers of PTA need only add a suitable amount of HBr in production to supplement Br required for catalysis.

The manufacturers of PTA must also satisfy environmental requirements as well as industrial safety requirements when they unload and inject HBr. Additionally, adding HBr to the process is another manpower expense. Catalyst suppliers are therefore requested to mix HBr with CMA in catalysts. This mixture is the most widely used “brominated cobalt manganese acetate in solution” on the market and is called Co—Mn—Br catalyst solution (CMB). However, solutions of HBr contain free bromine molecules, which are highly corrosive, and free acid, which has strong acidity. These compounds are very dangerous to handle and are dangerous to the environment.

Metallic equipment in PTA factories is adversely effected when storing and producing PTA because of the corrosive characteristics of HBr. Unfortunately, the exclusive use of Br in the oxidation catalysts for manufacturing PTA remains inevitable with the current technology. Therefore, the industry desires compounds other than HBr to avoid the problems incurred when CMB solution is used as the catalyst.

### SUMMARY OF THE INVENTION

The invention is a preparation using CoBr₂ and MnBr₂ instead of HBr to achieve low corrosive characteristics when compared to standard sources of Br. Additionally, the CoBr₂ and MnBr₂ compounds of the invention provide a source of Co and Mn for the reaction process. For preparations of catalysts where the percent of Br is higher than (Co %×2.7)+(Mn %×2.9), the use of HBr is required to supplement Br in the process. However, this supplement still minimizes the corrosive nature of CMB used in the reaction process.

The invention includes a method for maintaining a catalytic solution system for manufacturing terephthalic acid. The method includes the step of conducting an oxidation reaction in a Co—Mn—Br acetate solution wherein the oxidation reaction consumes Co, Mn, and Br. Replenishing of the Co, Mn, and Br in the system is performed under one of two conditions. A first condition is when a weight ratio of each component is (Co %×2.7)+(Mn %×2.9%)>Br %, the Co and the Mn are replenished by cobalt acetate and manganese acetate produced by the Co—Mn—Br acetate solution and a source of CoBr₂ and a source of MnBr₂ solely replenish the Br. A second condition is when a weight ratio of each component is (Co %×2.7)+(Mn %×2.9%)<Br %, the Co and the Mn are replenished by cobalt acetate and manganese acetate produced by the Co—Mn—Br acetate solution and a source of CoBr₂ and a source of MnBr₂ replenish a majority of the Br with a remainder of the consumed Br being replaced by a source of HBr.

The invention is used in chemical reactions of para-xylene with gas containing oxygen to manufacture terephthalic acid. The invention greatly improves the performance of the catalyst solution.

The object of the invention is to provide a Co—Mn—Br catalyst solution (CMB) for manufacturing terephthalic acid. The solution has the effect of lowering the corrosive nature of CMB and increasing storage and operating safety as well as improving the quality of PTA product.

The invention achieves this objective by providing a catalytic solution system for manufacturing terephthalic acid. The system includes a Co—Mn—Br acetate solution (CMB) for use in an oxidation reaction such that when a weight ratio of each component is (Co %×2.7)+(Mn %×2.9%)>Br %, only CoBr₂ and MnBr₂ are introduced as a source of Br in the CMB system. The system is absent from the highly corrosive HBr.

The invention as well as the effect achieved thereof will be apparent from the features and technical content of the detailed description of the preferred embodiment and the accompanying drawing.

### BRIEF DESCRIPTION OF THE DRAWINGS

FIG. 1 is a comparative chart on the static corrosive characteristics of the CMB solution of the invention compared to other CMB solutions.

### DETAILED DESCRIPTION OF THE PREFERRED EMBODIMENT

The invention is a catalytic solution system for manufacturing terephthalic acid including a Co—Mn—Br acetate solution (CMB), wherein manganese bromide (MnBr₂) and cobalt bromide (CoBr₂) solutions are directly mixed to form a Co—Mn—Br catalyst solution (CMB). When an addition of a Br source is required and a mixed concentration of Co and Mn is not exceeded, MnBr₂ and CoBr₂ are used. The use of HBr, which is very corrosive, is avoided.

Purified terephthalic acid (PTA) manufacturing processes often experience an accumulation of metal residues caused by corrosion of the equipment and of organics caused by side reactions. These concentrations must be controlled by quantitative discharges of the “mother solution.” These discharges expel part of Br together with the gas formed by the reaction, such as Br₂ and CH₃Br. The discharges also expel part of the Co and Mn, which flow out with the residue purged from mother liquid. Therefore, it is necessary to supplement fresh CMB solution in order to maintain the required concentration for the reaction. The supplement of compounds for the PTA manufacturing processes is calculated by the degree of loss of Co, Mn, and Br from the system.

The invention is described in detail by the following embodiment, but it is not intended to be limited to the embodiment.

The preparation of the catalyst of the invention mixes cobalt bromide solution and manganese bromide solution directly to obtain the desired Br percentage for a desired CMB. If the Br percentage is lower than (Co %×2.7)+(Mn %×2.9), the shortage of Co percent concentration and Mn percent concentration is then supplemented by cobalt acetate and manganese acetate. Under this condition no free acid and/or no free bromine is required for the supplement. When the Br percentage of CMB is higher than (Co %×2.7)+(Mn %×2.9), the shortage of Br is supplemented with HBr.

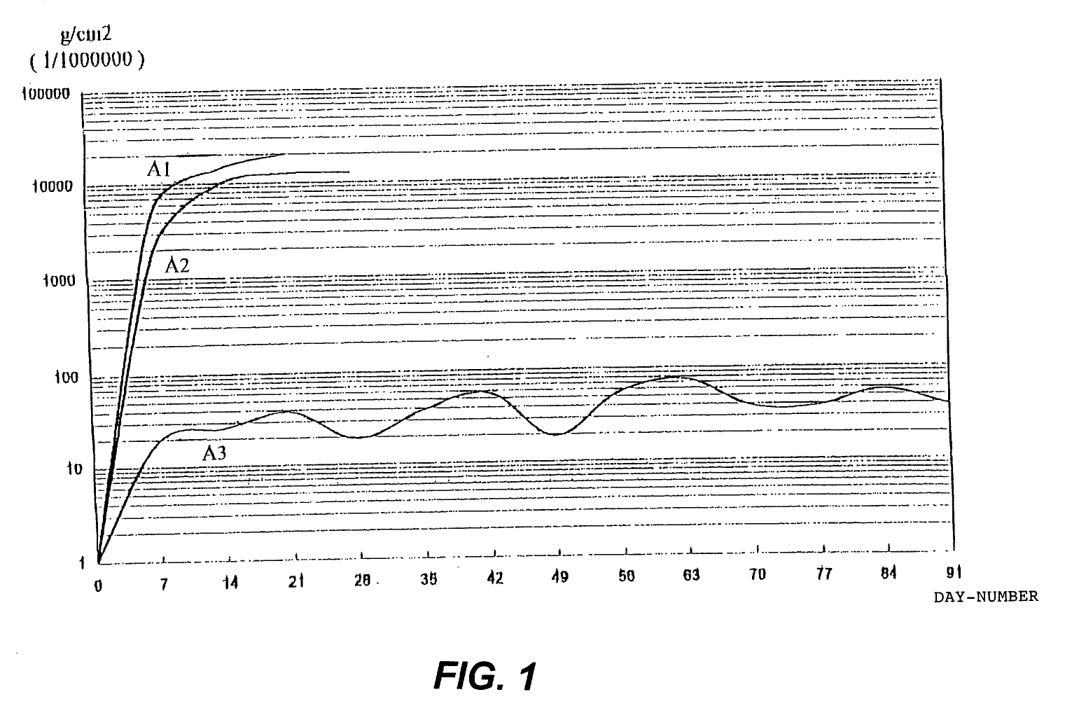

FIG. 1 illustrates the benefit of the invented catalyst system to PTA manufacturers. FIG. 1 illustrates the comparative corrosive characteristics of the invention and other catalyst systems.

FIG. 1 presents a test chart of different solutions. The curve “A1” represents the CMB solution using 100 percent HBr. The curve “A2” represents the CMB solution of which Br is obtained partly from MnBr₂/CoBr₂ and partly from HBr. The curve “A3” represents the CMB solution of which Br is obtained only from the bromide compounds of CoBr₂/MnBr₂.

The curves of the chart demonstrate that, when the desired Br is completely obtained from HBr, such a solution reaches its highest corrosive power within 14 to 21 days. When the Br percent is higher than (Co %×2.7)+(Mn %×2.9), HBr is accordingly added for the supplement. As is shown by the curve “A2,” the peak of the corrosive value reaches its highest value within 14 to 21 days also, but the corrosive extent is less than that exhibited by the curve “A1.”

The curve “A3” demonstrates that, when Br is obtained only from the bromide compound, the corrosive value of its CMB solution is lowered mostly up to 90 days. The corrosive extent is still at the lowest position. The present invention, therefore, can effectively improve the corrosive characteristic of CMB and increase the safety of operating and storing the catalyst in factories as well as improve production quality.

The embodiment stated above is only for illustrating the invention and not for limiting the scope of the invention. It will be apparent to those skilled in this art that various modifications or changes can be made to the elements of the invention without departing from the spirit and scope of this invention. Accordingly, all such modifications and changes also fall within the scope of the appended claims and are intended to form part of this invention.

1. A catalytic solution system for manufacturing terephthalic acid, the system comprises:
a Co—Mn—Br acetate solution for an oxidation reaction, the oxidation reaction consumes Co, Mn, and Br; and
a source of CoBr₂ and a source of MnBr₂, the source of CoBr₂ and a source of MnBr₂ solely replenish the Br when a weight ratio of each component is (Co %×2.7)+(Mn %×2.9%)>Br %, and Co and Mn are replenished by cobalt acetate and manganese acetate produced by the Co—Mn—Br acetate solution.

2. A catalytic solution system for manufacturing terephthalic acid, the system comprises:
a Co—Mn—Br acetate solution for an oxidation reaction, the oxidation reaction consumes Co, Mn, and Br; and
a source of CoBr₂ and a source of MnBr₂, the source of CoBr₂ and a source of MnBr₂ replenish a majority of the Br when a weight ratio of each component is (Co %×2.7)+(Mn %×2.9%)<Br %, and Co and Mn are replenished by cobalt acetate and manganese acetate produced by the Co—Mn—Br acetate solution, a remainder of the consumed Br is replaced by a source of HBr.

3. A method for maintaining a catalytic solution for manufacturing terephthalic acid, the method comprises:
conducting an oxidation reaction in a Co—Mn—Br acetate solution wherein the oxidation reaction consumes Co, Mn, and Br; and
replenishing Co, Mn, and Br in the system under one of two conditions,
a first condition being when a weight ratio of each component is (Co %×2.7)+(Mn %×2.9%)>Br %, the Co and the Mn are replenished by cobalt acetate and manganese acetate produced by the Co—Mn—Br acetate solution and a source of CoBr₂ and a source of MnBr₂ solely replenish the Br; and
a second condition being when a weight ratio of each component is (Co %×2.7)+(Mn %×2.9%)<Br %, the Co and the Mn are replenished by cobalt acetate and manganese acetate produced by the Co—Mn—Br acetate solution and a source of CoBr₂ and a source of MnBr₂ replenish a majority of the Br with a remainder of the consumed Br being replaced by a source of HBr.

4. The method of claim 3 further comprising:
stirring the catalytic solution by an action of para-xylene with a gas containing oxygen.
